(19) United States
(12) Patent Application Publication
(10) Pub. No.: US 2026/0345562 A1
(43) Pub. Date: Nov. 5, 2026

(54) PHARMACEUTICAL COMPOSITION FOR THE TREATMENT OF OCULAR NEOVASCULARISATION

(71) Applicant: NOVALIQ GMBH, Heidelberg (DE)

(72) Inventors: Heping XU, Belfast (GB); Frank DAUTZENBERG, Konstanz (DE); Madlen WITT, Heidelberg (DE)

(21) Appl. No.: 19/274,157

(22) Filed: Jul. 18, 2025

Related U.S. Application Data
(63) Continuation of application No. 17/424,489, filed on Jan. 17, 2020, now abandoned.

(30) Foreign Application Priority Data
Jan. 21, 2019 (EP) 19152839.7

(51) Int. Cl. A61K 31/436; A61K 9/00; A61K 31/02; A61P 27/02

(52) U.S. Cl. CPC A61K 31/436; A61K 9/0048; A61K 31/02; A61P 27/02

(57) ABSTRACT

The present invention relates to a pharmaceutical composition comprising tacrolimus and a semifluorinated alkane for use in the treatment of ocular neovascularisation.

### FIELD OF THE INVENTION

The present invention relates to a pharmaceutical composition comprising tacrolimus for use in the treatment of ocular neovascularisation. Moreover, the present invention relates to a method of treatment of ocular neovascularisation comprising administering a composition comprising tacrolimus.

### BACKGROUND OF THE INVENTION

Angiogenic eye diseases are among the most common causes of blindness worldwide. Angiogenesis is controlled by a dynamic equilibrium between proangiogenic and anti-angiogenic factors. Several circumstances can influence the balance and lead to neovascularisation. Ocular angiogenesis, particular in the retina and in the choroid, may lead to significant visual impairment. Diabetic retinopathy, neovascular age related macular degeneration (AMD), retinopathy of prematurity and retinal vessel occlusion are major causes of angiogenesis related vision loss.

Vascular endothelial growth factor (VEGF) is considered the most critical regulator of ocular angiogenesis. Currently, important anti-VEGF therapies include bevacizumab, aflibercept, ranizumab and others. However, current treatment approaches are insufficiently effective and partially associated with significant adverse effects. Some patients may present worsening of the eye disease, suggesting that other vascular mediators may also contribute to ocular angiogenesis. (Cabral et al., Int. J. Retin. Vitr. (2017) 3:31).

Other approaches currently favour or even require the systemic administration of substances like somatostatin analogues and PKC-inhibitors. However, the systemic administration of bioactive substances always risks significant systemic adverse effects. Due to the morphological characteristics of the eye, local therapies including intraocular injection or even local gene transfer might be feasible. (Wegewitz et al., Curr Pharm Des. 2005; 11 (18): 2311-30.)

Tacrolimus (FK506), a macrolide lactone isolated from fungus Streptomyces tsukubaensis, is a potent immunosuppressive drug, used mainly after organ transplant to lower the risk of organ rejection. Tacrolimus is also used to suppress the inflammation associated with ulcerative colitis or as an ointment in the treatment of eczema. It has been reported that tacrolimus has a poor ability to penetrate tissue barrier upon topical administration due to its physicochemical properties (Tamura et al., 2002, J. Pharm. Sci. 91, 719-729).

### SUMMARY OF THE INVENTION

It is the problem of the present invention to provide a pharmaceutical composition which provides an effective treatment for ocular neovascularisation and overcomes the drawbacks associated with the current treatments.

This problem is solved according to the present invention by a pharmaceutical composition comprising tacrolimus and a semifluorinated alkane.

The present invention is based on the recognition that tacrolimus has anti-angiogenic roles and tacrolimus in semifluorinated alkane eyedrop has therapeutic potential for angiogenic eye diseases.

According to a first aspect, the present invention provides a pharmaceutical composition comprising tacrolimus and a semifluorinated alkane for use in a method of treatment of ocular neovascularisation.

In a second aspect, the present invention provides a method of treating ocular neovascularisation by administering a pharmaceutical composition comprising tacrolimus and a semifluorinated alkane to the eye of a subject in need thereof.

Further, the present invention provides a pharmaceutical composition comprising tacrolimus and a semifluorinated alkane for the manufacture of a medicament for use in the treatment of ocular neovascularisation.

### DETAILED DESCRIPTION OF PREFERRED EMBODIMENTS

The term “semifluorinated alkane” (also referred to as “SFA” throughout this document) as used herein refers to a linear or branched compound composed of at least one perfluorinated segment (F-segment) and at least one non-fluorinated hydrocarbon segment (H-segment). Preferably, the semifluorinated alkane is a linear or branched compound composed of one perfluorinated segment (F-segment) and one non-fluorinated hydrocarbon segment (H-segment). Preferably, said semifluorinated alkane is a compound that exists in a liquid state within the temperature range of 4° to 40 °C. In one embodiment, the perfluorinated segment and/or the hydrocarbon segment of the said SFA optionally comprises or consists of a cyclic hydrocarbon segment, or optionally said SFA comprises an unsaturated moiety within the hydrocarbon segment.

It is preferred that the F- and the H-segment of the linear or branched semifluorinated alkane comprise, independently from one another, 2 to 10 carbon atoms.

According to a preferred embodiment of the present invention, the semifluorinated alkane is a linear compound of the formula (I) CF₃(CF₂)ₙ(CH₂)ₘCH₃, wherein n and m are integers independently selected from each other from the range of 2 to 10, preferably selected from the range of 2 to 8 and even more preferably selected from the range of 3 to 7.

Optionally, the linear or branched SFA may comprise a branched non-fluorinated hydrocarbon segment comprising one or more alkyl groups selected from the group consisting of —CH3, —C2H5, —C3H7 and —C4H9 and/or the linear or branched SFA may comprise a branched perfluorinated hydrocarbon segment, comprising one or more perfluorinated alkyl groups selected from the group consisting of —CF3, —C2F5, —C3F7 and —C4F9.

According to another nomenclature, the linear semifluorinated alkane may be referred to as FnHm, wherein F means the perfluorinated hydrocarbon segment, H means the non-fluorinated hydrocarbon segment and n, m is the number of carbon atoms of the respective segment. For example, F4H5 is used for 1-perfluorobutyl-pentane.

In a preferred embodiment of the present invention, the semifluorinated alkane is at least one linear semifluorinated alkane of formula (I) wherein n is selected from 3 to 5 and m is selected from 4 to 7. Preferably, the semifluorinated alkane is one selected from F4H5 and F6H8. More preferably, the semifluorinated alkane is F4H5.

The present pharmaceutical composition according to the present invention may comprise more than one SFA. It may be useful to combine different SFA's, for example, in order to achieve a particular target property such as a certain density or viscosity. If a mixture of two or more different SFA's is used, it is furthermore preferred that the mixture comprises at least one of F4H5, F4H6, F6H4, F6H6, F6H8 and F6H10, and in particular one of F4H5, F6H6 and F6H8. In another embodiment, the mixture comprises at least two members selected from F4H5, F4H6, F6H4, F6H6, F6H8, and F6H10, and in particular at least two members selected from F4H5, F6H6 and F6H8.

Liquid SFAs are chemically and physiologically inert, colourless and stable. Their typical densities range from 1.1 to 1.7 g/cm³, and their surface tension may be as low as 19 mN/m. SFA's of the FnHm type are insoluble in water but also somewhat amphiphilic, with increasing lipophilicity correlating with an increasing size of the non-fluorinated segment.

The pharmaceutical composition for use according to the present invention comprises tacrolimus. Tacrolimus may be present in the composition in an amount of at least 0.01% w/v based on the total volume of the composition, preferably in an amount of at least 0.02% w/v based on the total volume of the composition. In a preferred embodiment, tacrolimus is present in an amount of from 0.01% w/v to 0.1% w/v, preferably in an amount of from 0.01% to 0.05% w/v, more preferably in an amount of from 0.02% w/v to 0.05% w/v with respect to the total volume of the composition.

Unless otherwise indicated, the term “% w/v” as used throughout herein denotes the amount of a component of the composition as weight percentage in relation to the total volume of the composition (with “w” denoting the weight and “v” denoting the volume). For example, 0.02% w/v may be understood as relating to 0.2 mg in 1 mL of the composition.

In the pharmaceutical composition according to the present invention, tacrolimus may be dissolved or suspended in the semifluorinated alkane. Preferably, the pharmaceutical composition for the use of the present invention is in the form of a solution, more preferably in the form of a clear solution.

In one embodiment the pharmaceutical composition for the use of the present invention may comprise at least 85% wt, preferably at least 90% wt and more preferably at least 95% wt of the semifluorinated alkane, based on the total weight of the composition.

In a preferred embodiment, the semifluorinated alkane is present in an amount of from 96 to 99.5% percent by weight based on the total weight of the composition, preferably in an amount of from 98 to 99 percent by weight based on the total weight of the composition.

In the present invention the pharmaceutical composition may optionally comprise further solvents and excipients as described in detail below. The term “excipients” as used herein refers to any pharmaceutically acceptable natural or synthetic substance that may be added to the pharmaceutical composition of the present invention, to enhance or otherwise modify its physical or chemical constitution or stability or therapeutic properties. One or more excipients such as, for example, an antioxidant, a preservative, a lipid or oily excipient, a surfactant or a lubricant or a combination of at least 2 excipients thereof may be present in the pharmaceutical composition according to the present invention.

Suitable antioxidants for use in the present pharmaceutical composition comprise, for example: butylated hydroxytoluene (BHT), butylated hydroxyanisole (BHA), tertiary butylhydroquinone (TBHQ), vitamin E, vitamin E derivatives (i.e. alpha-tocopherol acetate) and/or ascorbic acid.

Suitable lipid or oily excipients for use in the pharmaceutical composition according to the present invention comprise, for example, triglyceride oils (i.e. soybean oil, olive oil, sesame oil, cotton seed oil, castor oil, sweet almond oil), triglycerides, mineral oil (i.e. petrolatum and liquid paraffin), medium chain triglycerides (MCT), oily fatty acids, isopropyl myristate, oily fatty alcohols, esters of sorbitol and fatty acids, oily sucrose esters, or any other oily substance which is physiologically tolerated by the eye.

Suitable lubricants for use in the pharmaceutical composition according to the present invention comprise, for example, carboxymethylcellulose and its sodium salt (CMC, carmellose), polyvinyl alcohol, hydroxypropyl methylcellulose (HPMC, hypromellose), hyaluronic acid and its sodium salt, and hydroxypropyl guar gum.

The pharmaceutical composition according to the present invention may or may not comprise pharmaceutically suitable natural or synthetic preservatives, such as, for example, benzalkonium chloride and chlorhexidine. In a preferred embodiment, however, the pharmaceutical composition according to the present invention does not comprise a pharmaceutically acceptable preservative.

In addition to the excipients as described above as optional components, the pharmaceutical composition according to the present invention may also comprise one or more further solvents. The term “further solvents” as used herein refers to a solvent or mixture of two or more different solvents other than the semifluorinated alkane. Suitable further solvents may be chosen from, for example, alcohols, such as ethanol, isopropanol or other further solvent which is physiologically tolerated by the eye.

A preferred solvent is ethanol which may be present in the pharmaceutical composition for use according to the present invention in an amount of up to about 2 percent by weight, preferably up to about 1.5 percent by weight based on the total weight of the composition. More preferably, the pharmaceutical composition for use according to the present invention comprises between 0.5 and 1.5 percent by weight of ethanol based on the total weight of the composition. Most preferably, the pharmaceutical composition for use according to the present invention comprises ethanol in an amount from 1.0 to 1.5 percent by weight based on the total weight of the composition. In an even more preferred embodiment, ethanol is present at a concentration of 1.4 percent by weight based on the total weight of the composition.

The term “% wt” as used herein and unless indicated otherwise refers to the amount of a component of a composition as a weight percentage in relation to the total weight of the pharmaceutical composition. The same meaning has to be given to “% w/w”, with w indicating the weight.

In a further embodiment, water can also be present in the pharmaceutical composition according to the present invention, however, preferably in small amounts of up to 1.0 wt.-% or even up to 0.1 wt.-% or less, based on the total weight of the composition. In a preferred embodiment, the pharmaceutical composition according to the present invention is essentially free of water, whereas the residual water may be attributed to the potential residual water content of tacrolimus. The term ‘essentially’ as used herein means if present then in trace or residual amounts such as to confer no technical advantage or relevance in respect of the object of the invention.

In a preferred embodiment, the pharmaceutical composition for use according to the invention comprises tacrolimus at a concentration of from 0.01 to 0.1% w/v and a semifluorinated alkane selected from F4H5 and F6H8. In a more preferred embodiment, the pharmaceutical composition for use according to the invention comprises tacrolimus at a concentration of from 0.01 to 0.05% w/v and 1-perfluorobutylpentane.

The pharmaceutical composition for use according to the present invention may comprise from 0.01% to 0.05% (w/v) of tacrolimus, 0.5 to 1.5% (w/w) ethanol and a semifluorinated alkane selected from F4H5 (1-perfluorobutyl-pentane) and F6H8 (1-perfluorohexyl-octane).

Preferably, the pharmaceutical composition for use according to the present invention comprises from 0.01% to 0.05% (w/v) of tacrolimus, from 1.0% to 1.5% (w/w) ethanol and the semifluorinated alkane is F4H5; more preferably from 0.01% to 0.03% (w/v) of tacrolimus, from 1.0% to 1.5% (w/w) ethanol and the semifluorinated alkane is F4H5; most preferably 0.02% w/v tacrolimus, 1.4% (w/w) ethanol and a semifluorinated alkane which is F4H5 (1-perfluorobutyl-pentane).

The pharmaceutical composition according to the present invention is especially useful as an ophthalmic composition, and may preferably be administered topically to the eye, eye lid, eye sac, eye surface and/or to an ophthalmic tissue of a patient. Preferably, however, the pharmaceutical composition of the present invention may be topically administered to an outer surface of an eye of a patient or to an ophthalmic tissue which is readily accessible by the patient or by another person administering the pharmaceutical composition to the eye of the patient in need thereof.

The pharmaceutical composition for the use of the present invention may be administered topically or by intravitreal injection to the eye. Preferably, the pharmaceutical composition for the use of the present invention is administered topically to the eye of a patient in need thereof.

Depending on the extent of the disease or on whether or not both eyes of the patient to be treated are affected, the present pharmaceutical composition may be administered to only one eye or to both eyes of the patient. Preferably, the pharmaceutical composition according to the present invention is administered at a volume of about 5 to 30 μl per dose per eye, preferably with a volume of about 8 to 15 μl per dose per eye, more preferably with a volume of about 8 to 12 μl per dose per eye. In a preferred embodiment, the pharmaceutical composition for the use of the present invention is administered topically to the eye at a volume of from 8 to 12 μl.

The pharmaceutical composition for the use of the present invention may be administered in an amount of 0.5 to 10 microgram of tacrolimus per dose per eye, preferably from 1 to 6 micrograms, more preferably from 1 to 3 micrograms of tacrolimus per dose per eye. The pharmaceutical composition for the use of the present invention may be administered once daily to four times daily, preferably once or twice daily, more preferably twice daily.

The term “angiogenic eye diseases” as used herein refers to abnormal ocular angiogenesis which occurs in a broad spectrum of eye disorders, including retinal vessel occlusion, retinopathy of prematurity (ROP), diabetic retinopathy (DR), neovascular age-related macular degeneration (AMD), neovascular glaucoma, and corneal neovascularization secondary to chemical injury or infectious or inflammatory processes. Angiogenesis is the process where endothelial cells in existing vessels sprout and form new vessels under the guidance and balance of numerous angiogenic stimulators and inhibitors. Angiogenesis plays important roles in both physiologic development and pathologic events. Abnormal angiogenesis is associated with many diseases, including for example cancers, cardiovascular diseases, neurodegeneration, and proliferative retinopathies.

Neovascularisation within the eye causes blindness in several ocular diseases, the most common of which are proliferative diabetic retinopathy, neovascular age related macular degeneration and retinopathy of prematurity. In diabetic retinopathy and retinopathy of prematurity the neovascularisation emanates from the retinal vasculature and extends into the vitreous cavity. Age related macular degeneration is associated with neovascularisation originating from the choroidal vasculature and extending into the subretinal space (K. Neely et al., Ocular neovascularisation, American Journal of Pathology, vol. 153, No. 3, September 1998)

In the present invention the ocular neovascularisation may be retinal or choroidal neovascularisation, preferably choroidal neovascularisation. In a preferred embodiment, the ocular neovascularisation is associated with or caused from age related macular degeneration and/or retinopathy. In a more preferred embodiment, the ocular neovascularisation is associated with or caused from age related macular degeneration, preferably wet age related macular degeneration.

Models of retinal and choroidal angiogenesis, including oxygen-induced retinopathy, laser-induced choroidal neovascularization, and transgenic mouse models with deficient or spontaneous retinal/choroidal neovascularization, as well as models with induced corneal angiogenesis, are widely used to investigate the molecular and cellular basis of angiogenic mechanisms.

In a second aspect, the present invention provides for a method of treating ocular neovascularisation, comprising administering a pharmaceutical composition comprising tacrolimus and a semifluorinated alkane to the eye of a subject in need thereof.

It should be noted that for the method according to this aspect of the invention, all embodiments and preferred embodiments described above in connection with the other aspects of the invention apply respectively.

Preferred embodiments of the method according to this aspect of the invention are:
1. A method of treating ocular neovascularisation comprising administering a pharmaceutical composition comprising tacrolimus and a semifluorinated alkane to the eye of the subject in need thereof.
2. The method according to item 1, wherein the ocular neovascularisation is retinal and/or choroidal neovascularisation.
3. The method according to any of item 1 or 2, wherein the ocular neovascularisation is caused or associated with a disease selected from age related macular degeneration, diabetic macular edema, central retinal vein occlusion, retinopathy, branch retinal vein occlusion.
4. The method according to any of the preceding items, wherein the semifluorinated alkane is a semifluorinated alkane of formula (I)

CF₃(CF₂)n(CH₂)mCH₃ (I)

wherein n is an integer selected from 2 to 10 and m is an integer selected from 2 to 10.
5. The method according to any of the preceding item, wherein n is 3 and m is 4 or wherein n is 5 and m is 7.
6. The method according to any of the preceding item, wherein tacrolimus is present at a concentration of at least 0.01% w/v with respect to the total volume of the composition.
7. The method according to any of the preceding items, wherein tacrolimus is present at a concentration of from about 0.01% w/v to 0.1% w/v with respect to the total volume of the composition.
8. The method according to any of the preceding items, wherein the composition further comprises ethanol.
9. The method according to item 8, wherein ethanol is comprised at a concentration of from about 0.5 to 2% w/w with respect to the total weight of the composition.
10. The method according to any of the preceding items, wherein the composition is topically applied or intravitreously injected to the eye.
11. The method according to any of the preceding items, wherein the ocular neovascularisation is due to uncontrolled expression of pro-angiogenic factors by inflammatory cells.
12. The method according to any of the preceding items, wherein the dose of tacrolimus administered to the eye is from about 0.5 to 10 micrograms per dose per eye.
13. The method according to any of the preceding items, wherein the composition is in form of a solution.
14. The method according to any of items 1 to 12, wherein the composition is in form of a suspension.
15. The method according to item 14, wherein the semifluorinated alkane is F6H8.
16. The method according to any of items 1 to 13, wherein the semifluorinated alkane is 1-perfluorobutylpentane.
17. The method according to any preceding items, wherein tacrolimus is present at a concentration of from 0.01 to 0.05% w/v, preferably from 0.01 to 0.03% w/v, more preferably 0.02% w/v.
18. The method according to item 17, wherein the composition further comprises ethanol, preferably at a concentration of from 1.0 to 1.5% w/w, more preferably at a concentration of 1.4% w/w with respect to the total weight of the composition.

In a third aspect, the present invention provides a kit comprising a container for holding the pharmaceutical composition for the use of the present invention and a data carrier, wherein the container is adapted for topical application or intravitreal injection of said composition to the eye, and wherein the data carrier comprises instructions for use of said composition in a method for treatment of ocular neovascularisation.

Further provided is the use of a kit according to the third aspect of the present invention in a method for treatment of ocular neovascularisation.

It should be noted that for the kit and its use according to this aspect of the invention, all embodiments and preferred embodiments described above in connection with the other aspects of the invention apply respectively.

In the kit according to the invention, the container may hold a single dose or a plurality of doses of the pharmaceutical composition comprising tacrolimus and a semifluorinated alkane as described above.

Furthermore, the kit according to this aspect of the invention may further comprise instructions for use of the container for dropwise topical administration of the composition to a surface of the eye of a patient or for intravitreal injection. The instructions or directions for use preferably comprised by the kit according to this aspect of the invention may be in any form suited to instruct the user how to perform the topical administration or the intravitreal injection to the affected eye of the patient or subject. It may be in any readable or tangible form, preferably in printed form or in any machine- or computer-readable form preferably in form of a machine-readable optical label such as, for example, a barcode or a QR-code. In a particularly preferred embodiment the directions for use are provided in form of an instruction leaflet, product or package insert or as an enclosed label.

In a further aspect, the present invention provides for a pharmaceutical composition comprising tacrolimus and a semifluorinated alkane for the manufacture of a medicament for use in the treatment of ocular neovascularisation.

It should be noted that also for this aspect of the invention, all embodiments and preferred embodiments described above in connection with the other aspects of the invention apply respectively.

The present inventors also conducted an in vitro study using BMDMs (culture of bone marrow derived macrophages) which study suggests that Tacrolimus and Dexamethasone have different anti-angiogenic profiles. Tacrolimus can reduce the expression of pro-angiogenic growth factors, and dexamethasone can increase the expression of anti-angiogenic growth factors. Tacrolimus may be more effective in diseases that are related to uncontrolled expression of pro-angiogenic factors by inflammatory cells, whereas Dexamethasone may be more effective in diseases related to reduced expression of anti-angiogenic factors by immune cells.

### DETAILED DESCRIPTION OF THE DRAWINGS

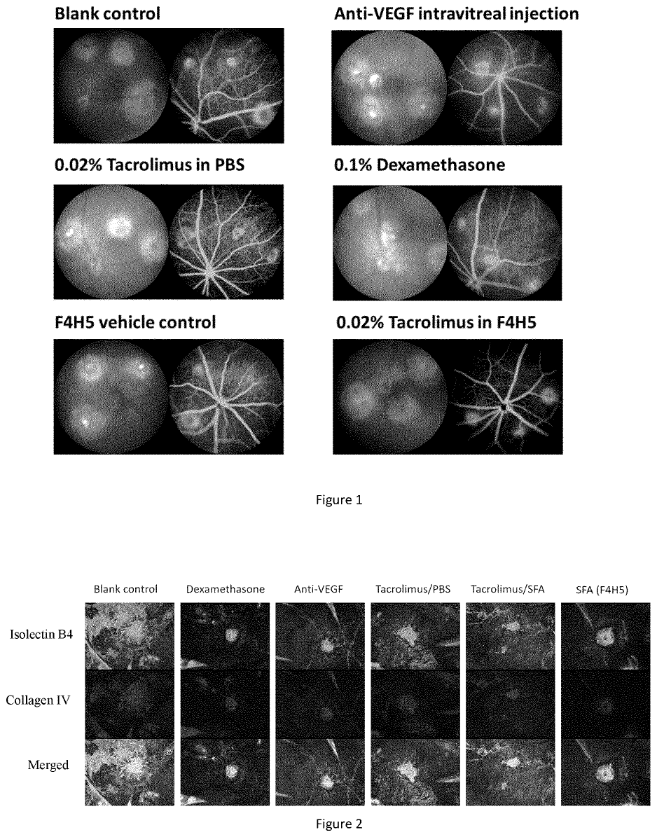

FIG. 1: CNV lesions in fundus images and FA images. 10 post-CNV induction mice were subjected to fundus examination and FA using the Micron IV system. Images showing are representatives from three mice in each group.

FIG. 2: The effect of different treatments on CNV formation. Lesion areas were quantified based on isolectin B4 staining. Representative RPE flatmount images showing CNV in each group.

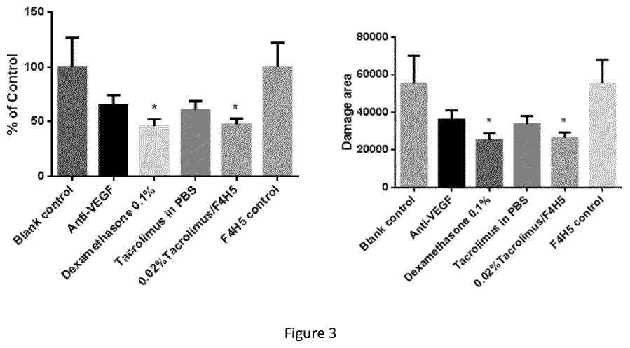

FIG. 3: the mean CNV size in each group expressed as either the absolute area or percentage of reduction compared with control non-treatment group. Data presented are Mean±SEM. * p <0.05, unpaired Student t test. Anti-VEGF, Dexamethasone 0.1% and Tacrolimus/PBS groups were compared to Blank control; 0.02% Tacrolimus/F4H5 was compared with F4H5 vehicle control.

### EXAMPLES

### Materials

The materials used to prepare the tacrolimus formulations listed below are: Tacrolimus (Euticals; Purity 98.2%), Ethanol (Merk, Seccosolv®, dried 0.01% H₂O), 1-perfluorobutyl-pentane F4H5 (Novaliq), phosphate buffered saline (VWR, biotechnological grade)

### Formulations

The following formulations were used in the experiments:

A solution of 0.02% w/v (0.2 mg/ml) Tacrolimus in 1.4% w/w Ethanol in 1-perfluorobutyl-pentane was prepared by dissolving tacrolimus in ethanol and 1-perfluorobutyl-pentane (this formulation is also referred to as Tacrolimus/SFA in Table 1, Table 2 and the Figures)

A suspension of Tacrolimus 0.02% w/v in phosphate buffered saline (PBS) was prepared (this formulation is also referred to as Tacrolimus/PBS in Table 1, Table 2 and the Figures)

Dexamethasone ophthalmic suspension Maxidex® 0.1% was purchased (Novartis Pharmaceuticals UK Ltd.).

Antimouse VEGF antibody purchased from R&D systems.

### Preparation of a Suspension of Tacrolimus in F6H8

The required amount of tacrolimus (purity 100.5%; water content 2.35%) is weighed and then transferred into a grinding jar. The required volume of F6H8 is added to the jar, which is then placed into a ball mill (BM01) for one hour at 150 rpm and 10 minutes interval. The suspension is then separated from the balls by means of a pipette.

### Example 1

The therapeutic effect of eyedrops of Tacrolimus 0.02% w/v in ethanol 1.4% w/w and F4H5 was tested in a mouse model of laser-induced choroidal neovascularisation (CNV) and compared with the therapeutic effect of 0.1% Dexamethasone eyedrop and with intravitreal injection of anti-mouse VEGF, respectively.

### Study Design

36 female C57BL/6 mice (12 weeks old) were purchased from Harland Laboratories UK. All mice were housed and bred in a normal experimental room and exposed to a 12-hour dark 12-hour light cycle. All procedures concerning the use of animals in this study were performed according to the Association for Research in Vision and Ophthalmology (ARVO) Statement for the Use of Animals in Ophthalmic and Vision Research and under the regulations of the United Kingdom Animal License Act 1986 (UK).

### Laser-Induced Choroidal Neovascularisation (CNV)

The laser-induced CNV was conducted in C57BL/6 mice. Briefly, mice were anesthetized with intraperitoneal injection of 75 mg/kg ketamine and 7.5 mg/kg xylazine. The pupils were dilated with 1% tropicamide (Chauvin Pharmaceuticals Ltd, Essex, UK). Three 532 nm diode laser spots (200 mV, 100 msec, 100 μm) were applied to each fundus using Viscoelastic material Microvisc (Vision Matrix Ltd, Harrogate, UK) and a coverslip as a contact lens. The lesions were placed between retinal vessels 2 to 3 optic-disc diameter from the optic disc. Formation of a bubble at the site of laser application, which indicates rupture of Bruch's membrane, is an important factor in obtaining CNV, so only burns in which a bubble was produced were included in this study. The CNV lesion develops 2-3 days after laser treatment, peaks at day 7-12, and regresses after 14-20 days.

The laser induced choroidal neovascularisation protocol applied is also described in 1) Tobe, T., S. Ortega, J. D. Luna, H. Ozaki, N. Okamoto, N. L. Derevjanik, S. A. Vinores, C. Basilico, and P. A. Campochiaro. 1998. Targeted disruption of the FGF2 gene does not prevent choroidal neovascularization in a murine model. Am. J. Pathol. 153:1641-1646; 2) Toma, H. S., J. M. Barnett, J. S. Penn, and S. J. Kim. 2010. Improved assessment of laser-induced choroidal neovascularization. Microvasc. Res. 80:295-302.

### Treatment Regimens

Six experimental groups were included in this study. Six mice were used in each group (Table 1). Mice were treated with either eyedrops twice daily from day 0 to day 10 or intravitreal injection of 1 μL with a concentration of 1 ng/μL anti-VEGF immediately after CNV induction. During eyedrop administration, each eye received 5 μl of eyedrop.

TABLE 1
In vivo treatment groups and dosing details

| | No. of | | | Dosing |
|---|---|---|---|---|
| Group | animals | Treatment | Dose | route |
| Group 1 | 6 | None | N/A | N/A |
| Group 2 | 6 | 0.02% Tacrolimus/SFA | Twice daily | Eye drop |
| Group 3 | 6 | 0.02% Tacrolimus/PBS | Twice daily | Eye drop |
| Group 4 | 6 | Vehicle (SFA) | Twice daily | Eye drop |
| Group 5 | 6 | Anti-mouse VEGF | One | Intravitreal |
| | | (1 ng/1 μl/eye) | injection | |
| Group 6 | 6 | 0.1% dexamethasone | Twice daily | Eye drop |

### Clinical Examinations

The CNV lesions were examined clinically on day 10 post-CNV induction using the Micron IV (Phoenix Research Labs) system for colour fundus image and fluorescein angiography.

### Sample Collection

On day 10 post-CNV induction, all mice were sacrificed by CO₂ inhalation and eyes were carefully removed. Ocular tissue whole mounts were prepared using the following procedure. All eyes were fixed in 2% paraformaldehyde (Agar Scientific Ltd, Cambridge, UK) for 2 h at room temperature and then washed with PBS. To prepare retinal pigment epithelia (RPE)-choroidal whole mounts, the anterior segment of the eye including the cornea, ciliary body, iris and the lens were removed. Five vertical cuts were made in the eye cup, and the retinal tissue was then dissected off from the RPE/choroid. The extraocular tissues including conjunctiva and ocular muscles were carefully removed. The RPE/choroid whole mounts were then further processed for immunostaining.

### Immunostaining of RPE Flatmounts

RPE/choroidal whole mounts were permeabilised with 0.3% triton X-100 for 1 h at room temperature. The samples were then blocked with 6% BSA for a further hour and incubated with rabbit anti mouse collagen IV (1:100, AbD Serotec, Kidlington, UK) and Biotinylated isolectin B4 (1:100, Vector Laboratories Ltd, UK) overnight at 4 °C. After thoroughly washing (10 minutes×3), samples were incubated with FITC conjugated Streptavidin (Dako, Denmark) and tetramethylrhodamine goat anti-rabbit IgG (Invitrogen, UK) for 4 h at room temperature in dark. Finally, samples were washed and flatmouted on glass slides with Vectashield Mounting Medium (Vector Laboratories Ltd, Peterborough, UK) and observed by confocal microscopy. The samples were flatmounted on glass slides and examined by fluorescence microscopy (Leica DMI8).

### Image and Data Analysis

An imaging software ImageJ system was used to analyse the images. The green and red channels were analysed separately. To measure the size of the CNV, the border of the CNV was outlined manually and the size was automatically calculated. The average size of CNV in each group was expressed as mean±SEM. Student's t test (unpaired, two tails) was used to detect the difference between tacrolimus/dexamethasone treated group and relevant control groups. In addition, one way ANOVA Turkey's Multiple Comparison Test was also used to detect differences among different groups.

### Clinical Observations

Fundus examination and fluorescence angiography (FA) were conducted in three mice from each group on day 10 post-CNV induction. CNV lesions were detected as white spots in fundus images and hyper-fluorescent spots in FA (FIG. 1). All mice developed CNV lesions.

### Immunohistological Results

A total of 216 laser burns were applied in 72 eyes (36 mice). All laser burns showed a bubble and induced CNV. Table 2 and FIGS. 2 and 3 show the results of isolectin B4 labelling. The results show that 0.02% tacrolimus/SFA eyedrop and 0.1% dexamethasone eyedrop treatment significantly suppressed CNV.

TABLE 2
The average size of CNV in each
group as determined by isolectin B4 staining.

| | | Number | Mean CNV | |
|---|---|---|---|---|
| Group | Treatment | of CNV | (μm²) | SEM |
| 1 | None | 22 | 55394 | 14930 |
| 2 | 0.02% Tacrolimus/SFA | 31 | 26421 | 2919 |
| 3 | 0.02% Tacrolimus/PBS | 27 | 33837 | 4323 |
| 4 | Vehicle (SFA) | 27 | 55618 | 12391 |
| 5 | Anti mouse VEGF | 27 | 36008 | 5208 |
| 6 | 0.1% dexamethasone | 28 | 25336 | 3658 |

The 0.02% tacrolimus/F4H5 eyedrop showed anti-angiogenic effect in the mouse model of laser-induced CNV. The anti-angiogenic effect of 0.02% tacrolimus/F4H5 was similar to 0.1% Dexamethasone eyedrop when applied twice a day.

### Example 2

The antiangiogenic effect of Tacrolimus was also tested in an in vitro model of choroidal angiogenesis and compared with dexamethasone. The results of this test showed that tacrolimus at concentrations of 100 ng/mL, 20 ng/mL and 4 ng/mL suppressed choroidal angiogenesis but the effect was not dose-dependent. The suppressive effect was similar to the effect of 1 μM Dexamethasone.

1. A pharmaceutical composition comprising tacrolimus and a semifluorinated alkane for use in a method of treatment of ocular neovascularisation.

2. The composition for the use of claim 1, wherein the ocular neovascularisation is retinal and/or choroidal neovascularisation.

3. The composition for the use of claim 1 or 2, wherein the ocular neovascularisation is caused or associated with a disease selected from age related macular degeneration, diabetic macular edema, central retinal vein occlusion, retinopathy, branch retinal vein occlusion.

4. The composition for use of any of the preceding claims, wherein the semifluorinated alkane is a semifluorinated alkane of formula (I)
CF₃(CF₂)n(CH₂)mCH₃ (I)
wherein n is an integer selected from 2 to 10 and m is an integer selected from 2 to 10.

5. The composition for use of any of the preceding claims, wherein n is 3 and m is 4 or wherein n is 5 and m is 7.

6. The composition for use of any of the preceding claims, wherein tacrolimus is present at a concentration of at least 0.01% w/v with respect to the total volume of the composition.

7. The composition for use of any of the preceding claims, wherein tacrolimus is present at a concentration between about 0.01% w/v and 0.1% w/v with respect to the total volume of the composition.

8. The composition for use of any of the preceding claims, further comprising ethanol.

9. The composition for use of claim 8, wherein ethanol is comprised at a concentration of from about 0.5 to 2% w/w with respect to the total weight of the composition.

10. The composition for use of any of the preceding claims, wherein the composition is topically applied or intravitreously injected to the eye, preferably topically applied.

11. The composition for use of any of the preceding claims, wherein the ocular neovascularisation is due to uncontrolled expression of pro-angiogenic factors by inflammatory cells.

12. The composition for use of any of the preceding claims, wherein the dose of tacrolimus administered to the eye is from about 0.5 to 10 micrograms per dose per eye.

13. The composition for use according to any of the preceding claims, wherein the composition is in form of a solution.

14. The composition for use of any of claims 1 to 12, wherein tacrolimus is suspended in the semifluorinated alkane, preferably wherein tacrolimus is suspended in 1-perfluorohexyloctane.

15. A kit comprising a container for holding the composition for use of any of the claims 1 to 14 and a data carrier, wherein the container is adapted for topical application or intravitreal injection of said composition to the eye, and wherein the data carrier comprises instructions for use of said composition according to any of the claims 1 to 14.
